(19) United States
(12) Patent Application Publication
(10) Pub. No.: US 2008/0154116 A1
(43) Pub. Date: Jun. 26, 2008

(54) Method and apparatus for obtaining electrocardiogram (ECG) signals

(76) Inventors: G. Randy Duensing, Gainesville, FL (US); Charles A. Saylor, Gainesville, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/644,183

(22) Filed: Dec. 22, 2006

(51) Int. Cl. A61B 5/0402; A61B 5/055

(52) U.S. Cl. 600/410; 600/509

(57) ABSTRACT

Embodiments of the subject invention relate to a method and apparatus for obtaining an electrocardiogram (ECG) signal. Embodiments can separate a true ECG signal from one or more signals due to electric fields caused by moving electrical charges. In a specific embodiment, an ECG signal can be separated from one or more electric fields caused by blood flow. An embodiment pertains to a joint MRI and diagnostic ECG system. In an embodiment, the joint diagnostic quality ECG can add information to a MRI cardiac study. This additional information can be useful for MR guided intervention treatments, such as locating tissue that created bad electrical arrhythmia. In an embodiment, the subject method and apparatus can be utilized to obtain an ECG for patient located in a magnetic field of 1.5 T or higher, such as in MRI systems with 1.5 T or higher magnetic fields. Embodiments of the invention can use flow encoding with a changing magnetic field, with dense electrical sensors and inversion of the EEG data, utilizing this information to extract the flow related signals. Further, inversion to the source distribution of the flow related signals can be accomplished.

### BACKGROUND OF INVENTION

Electrocardiogram (ECG) signals are based on the surface potentials of the heart. It is desirable to obtain diagnostic quality ECG signals while a patient is being monitored in a magnetic resonance imaging (MRI) system. Current ECG with filtering on MRI systems only allows gating. Such ECG gating provides information regarding what part of the heart cycle the heart is at for purposes of triggering an MRI image to be taken at the desired point in the heart cycle. Furthermore, it is not presently possible to obtain adequate ECG quality on standard 1.5 T or higher MRI systems. In addition, ECG triggering can also be difficult on standard 3T or higher MRI systems. Accordingly, there is currently no diagnostic quality ECG system that can be used in the MRI system. The primary reason that it is not presently possible to obtain adequate ECG quality on standard 3T or higher MRI systems is the magneto-hydrodynamics (MHD) flow voltages, which are due to the flow of blood in the static magnetic field of the MRI system. The MHD flow voltages can have the same spectral characteristics as the true heart polarization signals and are thus difficult to extract. These MHD flow voltages are due to the flow of blood, which is a conductor, in a direction perpendicular to the static magnetic field, or other magnetic fields, of the MRI system. In fact, blood vessels can experience a force on them due to blood flow in the static magnetic field of the MRI system.

ECG leads pick up potential differences caused by the heart muscle's nerve control and also pick up potentials caused by any other electric fields. In order to obtain an ECG, electrodes (or leads) are placed on a patient's body, such as on the patient's arms, legs, and chest. The electrodes detect the electrical impulses generated by the heart and transmit them to the ECG machine. However, as discussed, the leads can also pick up potentials caused by any other electric fields. As an example, when the ECG is used in an MRI system, another set of electric fields are produced by the movement of conducting blood perpendicular to the static magnetic field. This effect is described by magneto-hydrodynamics (MHD). It can be difficult, if even possible, to separate the true ECG signal from the signals produced by the heart pushing the blood through the vessels around the heart. Accordingly, there is a need for a method and apparatus that can allow the separation of a true ECG signal from signals produced by the heart pushing blood through the vessels around the heart and other blood flow in the presence of a magnetic field, in order to enable diagnostic quality ECG.

Images can be accomplished with the MR scanner and these images, combined with the output ECG signals, can be used to create a three-dimensional (3D) representation of the surface of the patient's heart and/or to create a 3D representation of the electric potential of the surface of the heart. A dynamic 3D representation of the surface and/or electric potential of the surface of the heart when the heart is beating can also be produced with images of the heart and the output ECG signals. In addition, a blood flow map, in one, two, or three dimensions, can also be produced with images of the blood flow system of the patient and the output ECG signals.

### BRIEF SUMMARY

Embodiments of the subject invention relate to a method and apparatus for obtaining an electrocardiogram (ECG) signal. Embodiments can separate a true ECG signal from one or more signals due to electric fields caused by moving electrical charges. In a specific embodiment, an ECG signal can be separated from one or more electric fields caused by blood flow. An embodiment pertains to a joint MRI and diagnostic ECG system. In an embodiment, the joint diagnostic quality ECG can add information to a MRI cardiac study. This additional information can be useful for MR guided intervention treatments, such as locating tissue that created bad electrical arrhythmia. In an embodiment, the subject method and apparatus can be utilized to obtain an ECG for patient located in a magnetic field of 1.5 T or higher, such as in MRI systems with 1.5 T or higher magnetic fields. Embodiments of the invention can use flow encoding with a changing magnetic field, with dense electrical sensors and inversion of the EEG data, utilizing this information to extract the flow related signals. Further, inversion to the source distribution of the flow related signals can be accomplished.

Images can be accomplished with the MR scanner and these images, combined with the output ECG signals, can be used to create a three-dimensional (3D) representation of the surface of the patient's heart and/or to create a 3D representation of the electric potential of the surface of the heart. A dynamic 3D representation of the surface and/or electric potential of the surface of the heart when the heart is beating can also be produced with images of the heart and the output ECG signals. In addition, a blood flow map, in one, two, or three dimensions, can also be produced with images of the blood flow system of the patient and the output ECG signals.

### DETAILED DISCLOSURE

Embodiments of the subject invention relate to a method and apparatus for obtaining an electrocardiogram (ECG) signal. Embodiments can separate a true ECG signal from one or more signals due to electric fields caused by moving electrical charges. In a specific embodiment, an ECG signal can be separated from one or more electric fields caused by blood flow. An embodiment pertains to a joint MRI and diagnostic ECG system. In an embodiment, the joint diagnostic quality ECG can add information to a MRI cardiac study. This additional information can be useful for MR guided intervention treatments, such as locating tissue that created bad electrical arrhythmia. In an embodiment, the subject method and apparatus can be utilized to obtain an ECG for patient located in a magnetic field of 1.5 T or higher, such as in MRI systems with 1.5 T or higher magnetic fields. Embodiments of the invention can use flow encoding with a changing magnetic field, with dense electrical sensors and inversion of the EEG data, utilizing this information to extract the flow related signals. Further, inversion to the source distribution of the flow related signals can be accomplished.

Images can be accomplished with the MR scanner and these images, combined with the output ECG signals, can be used to create a three-dimensional (3D) representation of the surface of the patient's heart and/or to create a 3D representation of the electric potential of the surface of the heart. A dynamic 3D representation of the surface and/or electric potential of the surface of the heart when the heart is beating can also be produced with images of the heart and the output ECG signals. In addition, a blood flow map, in one, two, or three dimensions, can also be produced with images of the blood flow system of the patient and the output ECG signals.

In an embodiment of the subject method and apparatus, at least 4 ECG leads are placed on the patient. In a further embodiment, at least 60 ECG leads are used. As the heart produces time-varying potentials, and each ECG lead picks up the net voltage from the heart from the ECG lead's perspective, more detail can be provided when more ECG leads are used. In a specific embodiment, an electrode vest similar to the electrode vest taught by, and shown in FIG. 1a of, “Electrocardiographic imaging (ECGI): a new noninvasive imaging modality for cardiac electrophysiology and arrhythmia”, Yoram Rudy, Proc. of SPIE Vol. 6143, which is hereby incorporated by reference in its entirety, can be used. This vest uses 224 electrodes to produce 224 body-surface electrocardiograms. Other configurations of ECG electrodes can be used in accordance with various embodiments of the invention.

The electric field caused by a moving electric charge in a magnetic field, is proportional to the cross product of the velocity of the charge and the magnetic field. Equation (1) reflects this relationship, where V_{B} is the velocity of the blood, B_{S} is the static magnetic field, and E_{S} is the electric field caused by the flowing blood.

V_{B}×B_{S}=E_{S} (1)

Therefore, the potentials received at ECG electrodes due to the movement of blood are proportional to the velocity of the blood flow and the magnitude of the magnetic field perpendicular to the flow of the blood. The “true” ECG signal is not related to either the magnetic field or to the blood flow velocity. When measuring the heart, the frequency characteristics of the signal to be measured (ECG signal) are very similar to the frequency characteristics of the signal picked up by the ECG leads due to the flow of the blood. Accordingly, modulation of the magnetic field in the direction of the static magnetic field can provide additional information to allow the separation of the true ECG signal from the signal produced by the flow of the blood in a direction perpendicular to the static magnetic field. Equation (2) reflects the relationship of equation (1), with modulation of the static magnetic field, ΔB_{S}.

V_{B}B_{S}+V_{B}ΔB_{S}=Eₛ+ΔEₛ (2)

Preferably, the modulation is at a frequency that allows the blood flow signal to be separated from the bandwidth of the ECG signal during processing of the signal. A variety of modulation envelopes can be used for the magnetic field parallel to the static magnetic field, such as sinusoidal, ramp, square, or triangular. The magnitude of the modulation should be large enough to allow the separation of the blood flow signal due to the modulation of the static magnetic field from the ECG signal during processing of the signal. In a preferred embodiment, the magnitude of the modulation produces a change in the magnitude of the magnetic field in the direction of the static magnetic field of at least 0.5% of the magnitude of the static magnetic field, and more preferably at least 1.0% of the magnitude of the static magnetic field.

Embodiments of the invention can separate the “true” ECG signal from the blood flow induced potentials (magneto-hydrodynamic voltages) by modulating the static magnetic field so as to modulate the magneto-hydrodynamic voltages produced in the ECG sensors. In an embodiment, all vector components of the magnetic field can be modulated in a like manner. In an embodiment, for the same perturbation, the components perpendicular to the main field are larger than the components parallel to the main field. As an example, fields perpendicular to the static magnetic field of the MR scanner and in a first plane can be modulated. This can allow determination of blood flow in another dimension. Further, fields perpendicular to the static magnetic field of the MR scanner and in a second plane perpendicular to the first plane can be modulated allowing determination of blood flow in three dimensions. The determination of the blood can involve combining an image via the MR scanner of the patient's blood flow system with the information from the input ECG signals from the ECG leads. In a specific embodiment, measurements can be taken with the MR static magnetic field on and with the MR static magnetic field off, to produce additional information for providing a blood flow map. By correlating the signals at the ECG outputs with the modulation of the magnetic fields, the separation of the two signal types can be accomplished. In an embodiment, the modulation is at frequency ranges outside the cardiac frequency range, which is around 0.5 Hz to 20 Hz. In embodiments, the modulation can have frequency components either well below 0.5 Hz or well above 20 Hz. In embodiments, the modulation can have frequency components low enough such that after separation of the blood flow signal from the ECG signal the two signals can be distinguished from each other. In embodiments, the modulation can have frequency components high enough such that after separation of the blood flow signal from the ECG signal the two signals can be distinguished from each other. In addition, the frequency of the modulation should be selected to produce signals outside of the RF spectrum. Table I shows some typical MR frequencies.

TABLE I

| | MR Frequency in MHz | | |
|---|---|---|---|
| Isotope | 1T | 1.5T | 3T |
| ¹H | 42.6 | 63.9 | 127.7 |
| ²H | 6.5 | 9.8 | 19.6 |
| ¹³C | 10.7 | 16.1 | 32.1 |
| ¹⁴N | 3.1 | 4.6 | 9.2 |
| ¹⁷O | 5.6 | 8.4 | 16.7 |
| ¹⁹F | 40.1 | 60.1 | 120.2 |
| ²³NA | 11.3 | 16.9 | 33.8 |
| ³¹P | 7.2 | 10.9 | 21.7 |
| ³⁵Cl | 4.2 | 6.3 | 12.5 |
| ³⁹K | 2.0 | 3.0 | 6.0 |

In a specific embodiment, the true ECG signal can be extracted by modulating the magnetic field and separating the true ECG signal from the magneto-hydrodynamic induced signals when the MRI system is not active. This can avoid the need for further separation of voltages associated with gradients, and effects of changing the magnetic field on the spin system and thus the MR images being acquired. In another embodiment, the true ECG signal can be acquired continuously irrespective of the pulse sequence activity. In an embodiment involving continuous acquisition of the ECG signal, the frequency range used for modulation of the magnetic field is different than the frequencies in the pulse sequences used. In a specific embodiment, frequencies of 100 kHz or higher for the modulation of the magnetic field can be implemented. The modulation of the effective magnetic field can produce a copy of the spectrum of the MHD related signals in a frequency range that is free of other signals. Filtering for this can allow a model of the MHD only signals. Such filtering can be used to separate the MHD signals and ECG signals that share the same band, such as 1 Hz. The separation can be performed in approximate ways using techniques known in the art or performed in an exact manner if the strength of the perturbing field is known to a high accuracy. Multiple amplitudes of the perturbation can also be implemented to assist inversion. In a specific embodiment, for a 1.5T static magnetic field, with an RF frequency of approximately 64 MHz, a modulation frequency of between about 1 MHz and about 2 MHz can be used.

In an embodiment, changes in, or modulation of, the magnetic field is affected in all directions. The uniformity of the additional field can also be taken into consideration. The amplitude of the modulation can be substantial enough to produce sufficient changes in the voltages received by the ECG set, such that the signals can be separated. The currents in the windings producing these fields can also directly create voltages in the ECG leads and can be accounted for, for example, by extraction in the same way as the MHD effects. In another embodiment, another magnetic field can be modulated with respect to the velocity of the blood by moving the patient with respect to the magnetic field. This may be undesirable in certain situations during the actual scan, but can be useful in the magnet when the spin system is not active. In an embodiment, the separation is accomplished by performing a correlation of each signal with the modulation waveform and removing any strongly correlated components.

In an embodiment, gradient windings and/or shim coils can be used to provide the changes in, or modulation of, the magnetic field. Accordingly, in an embodiment, the gradient windings and/or shim coils of an MRI scanner can be used such that additional coils are not needed. In further embodiments, additional coils can be positioned in the MR scanner to provide the modulated portion of the fields, such as the modulated portions of the field, perpendicular to the static magnetic field of the MR scanner.

In an embodiment, utilizing a dense electrode array around the heart can produce a 3D map of the source electric potentials, diagnostic quality ECG signals, and the blood flow velocity map of the vessels around the heart and/or the heart itself. In order to acquire a 3D blood flow map, the magnetic field is also modulated perpendicular to the static magnetic field, such that if the static magnetic field is considered to be in the z-direction, the magnetic field is also modulated in the x and y directions. This can allow a blood flow map of the vessels around the heart and/or the heart itself without the need to put dye in the arteries, as in MR angiography. Furthermore, this can allow the simultaneous acquisition of a blood flow map and an ECG with an MRI scan, so as to eliminate the need for a separate scan procedure under, for example, CT or ultrasound. To accomplish a three-dimensional representation of the surface of the heart, images of the heart from the MR scanner can be combined with information from the output ECG signals, where the output ECG signals are the input ECG signals received from the ECG leads during modulation of the static magnetic field with the conductor flow related portion of the input ECG signals removed.

In an embodiment, a voltage pick-up antenna can be utilized to monitor the changes in, or modulation of, the magnetic field and/or to pick up voltages associated with gradient fields. These signals from the voltage pick-up antenna can be used to calibrate the system and further improve the accuracy of the true ECG signal computation. In another embodiment, electrodes can be utilized in pairs with opposing polarity to reduce the effect of voltage induction by non-local and relatively uniform sources, such as the changes in, or modulation of, the magnetic field used as the flow mapping field and gradient wave forms.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

1. A method of obtaining an electrocardiogram in the presence of a magnetic field, comprising:
positioning at least one electrocardiogram (ECG) lead on a patient in a region of interest in a static magnetic field;
modulating the static magnetic field in the region of interest;
receiving a corresponding at least one input ECG signal via the at least one ECG lead while modulating the static magnetic field in the region of interest;
determining a corresponding at least one output ECG signal from the at least one input ECG signal and the modulation of the static magnetic field in the region of interest.

2. The method according to claim 1, wherein positioning of at least one ECG lead on a patient in a region of interest in a static magnetic field comprises positioning at least four ECG leads on the patient in the region of interest in the static magnetic field.

3. The method according to claim 1, wherein positioning of at least one ECG lead on a patient in a region of interest in a static magnetic field comprises positioning at least sixty ECG leads on the patient in the region of interest in the static magnetic field.

4. The method according to claim 3, further comprising:
imaging the patient via magnetic resonance imaging in the region of interest.

5. The method according to claim 4, wherein imaging the patient comprises imaging the patient's heart, further comprising:
generating a three-dimensional representation of the electric potential of the surface of the patient's heart.

6. The method according to claim 5,
wherein the three-dimensional representation of the electric potential of the surface of the patient's heart is dynamic.

7. The method according to claim 4, wherein imaging the patient comprises imaging the patient's blood vessel system in the region of interest, further comprising:
generating a one-dimensional representation of the blood flow within the patient's blood flow system in the region of interest.

8. The method according to claim 1,
wherein determining at least one output ECG signal from the signal and the modulation of the static magnetic field in the region of interest comprises removing from the at least one input ECG signal components correlated to the modulation waveform.

9. The method according to claim 1,
wherein modulating the static magnetic filed comprises modulating the magnitude of the static magnetic field.

10. The method according to claim 1,
wherein determining a corresponding at least one output ECG signal comprises:
removing a corresponding at least one conductor flow signal from the at least one input ECG signal.

11. The method according to claim 10,
wherein the at least one conductor flow signal is at least one blood flow signal due to the patient's blood flowing in a direction perpendicular to the static magnetic field in the region of interest.

12. The method according to claim 1,
wherein the static field is modulated at frequencies below 0.5 Hz.

13. The method according to claim 1,
wherein the static field is modulated at frequencies about 20 Hz.

14. The method according to claim 1, further comprising:
modulating magnetic fields in a first plane and perpendicular to the static magnetic field in the region of interest, wherein the first plane is parallel to the static magnetic field.

15. The method according to claim 14, further comprising:
modulating magnetic field in a second plane and perpendicular to the static magnetic field in the region of interest, wherein the second plane is parallel to the static magnetic field and perpendicular to the first plane.

16. The method according to claim 14, further comprising:
generating a two-dimensional representation of the blood flow within the patient's blood flow system in the region of interest.

17. The method according to claim 15, further comprising;
generating a three-dimensional representation of the blood flow within the patient's blood flow system in the region of interest.

18. The method according to claim 9,
wherein the magnitude of the static magnetic field is modulated at least 0.5% during modulating the static magnetic field.

19. The method according to claim 9,
wherein the magnitude of the static magnetic field is modulated at least 1.0% during modulating the static magnetic field.

20. A method of obtaining an electrocardiogram during magnetic resonance imaging, comprising:
locating a patient in a region of interest of static magnetic field of an MR scanner;
positioning at least one electrocardiogram (ECG) lead on the patient in the region of interest;
imaging the patient in the region of interest via the MR scanner;
modulating the static magnetic field of the MR scanner in the region of interest;
receiving a corresponding at least one input ECG signal via the at least one ECG lead while modulating the static magnetic field in the region of interest;
determining a corresponding at least one output ECG signal from the at least one input ECG signal and the modulation of the static magnetic field of the MR scanner in the region of interest.

21. The method according to claim 20, wherein modulating the static magnetic field of the MR scanner comprises modulating the static magnetic of the MR scanner while imaging the patient in the region of interest.
